(19) United States
(12) Patent Application Publication
(10) Pub. No.: US 2012/0122933 A1
(43) Pub. Date: May 17, 2012

(54) Compounds

(76) Inventors: Ursula Hohlneicher, Molndal (SE); Silke Klick, Molndal (SE); Karin Lövqvist, Molndal (SE)

(21) Appl. No.: 13/102,387

(22) Filed: May 6, 2011

Related U.S. Application Data
(63) Continuation of application No. 11/570,748, filed on Dec. 15, 2006, now abandoned.
(60) Provisional application No. 60/582,619, filed on Jun. 24, 2004.

(51) Int. Cl. A61K 31/4439; A61P 1/00; A61P 29/00; C07D 401/12

(52) U.S. Cl. 514/338; 546/273.7

(57) ABSTRACT

The present invention relates to a novel crystalline form of esomeprazole sodium salt. Further, the present invention also relates to the use of the novel crystalline form for the treatment of gastrointestinal disorders, pharmaceutical compositions containing it as well as processes for the preparation of the novel crystalline form.

### FIELD OF THE INVENTION

The present invention relates to a novel crystalline form of esomeprazole sodium salt. Further, the present invention also relates to the use of the novel crystalline form for the treatment of gastrointestinal disorders, pharmaceutical compositions containing it as well as processes for the preparation of the novel crystalline form.

### BACKGROUND OF THE INVENTION AND PRIOR ART

Omeprazole, i.e. the compound 5-methoxy-2-[[(4-methoxy-3,5-dimethyl-2-pyridinyl)methyl]sulfinyl]-1H-benzimidazole, and therapeutically acceptable salts thereof, are described in EP 5129. Some specific alkaline salts of omeprazole are disclosed in EP 124 495.

Omeprazole is a sulfoxide and a chiral compound, wherein the sulfur atom is the stereogenic center. Thus, omeprazole is a racemic mixture of its two single enantiomers, the R- and S-enantiomer of omeprazole, the latter having the generic name esomeprazole. Esomeprazole is recently launched as a new generation of proton pump inhibitors, wherein the active pharmaceutical ingredient is esomeprazole magnesium salt. Esomeprazole shows improvements in the treatment of GERD compared to previous medications.

The absolute configurations of the enantiomers of omeprazole have been determined by an X-ray study of an N-alkylated derivative of the (+)-enantiomer in non-salt form. The (+)-enantiomer of the non-salt form and the (−)-enantiomer of the non-salt form were found to have R and S configuration, respectively, and the (+)-enantiomer of the magnesium salt and the (−)-enantiomer of the magnesium salt were also found to have R and S configuration, respectively. The conditions for the optical rotation measurement for each of these enantiomers are described in WO 94/27988.

Certain salts of single enantiomers of omeprazole and their preparation are disclosed in WO 94/27988. These compounds have improved pharmacokinetic and metabolic properties, which will give an improved therapeutic profile such as a lower degree of interindividual variation.

WO 96/02535 discloses a process for the preparation of the single enantiomers of omeprazole and salts thereof, including a sodium salt.

WO 98/54171 discloses a process for the preparation of the magnesium salt of the S-enantiomer of omeprazole trihydrate, wherein a potassium salt of S-omeprazole is used as an intermediate.

WO 00/44744 discloses a potassium salt of S-omeprazole free from methanol.

WO 03/089408 (Sun Pharmaceutical Industries Limited) discloses alkali or alkaline earth metal salts of esomeprazole, including a sodium salt.

### BRIEF DESCRIPTION OF THE DRAWINGS

FIG. 1 is an X-ray powder diffractogram of esomeprazole sodium salt modification B

### DESCRIPTION OF THE INVENTION

It has surprisingly been found that esomeprazole sodium salt can exist in more than one crystal form. The novel crystal form for the first time disclosed is hereinafter referred to as esomeprazole sodium salt modification B.

It is thus an object of the present invention to provide a crystalline form of esomeprazole sodium salt with advantageous properties.

It is an aspect of the present invention to provide esomeprazole sodium salt modification B.

Esomeprazole sodium salt modification B is characterized in providing an X-ray powder diffraction pattern, exhibiting substantially the following main peaks with d-values; 20.4, 14.0, 10.2, 8.8, 6.2, and 4.10 Å

The peaks, identified with d-values calculated from the Bragg formula and intensities, have been extracted from the diffractogram of esomeprazole sodium salt modification B. Only the main peaks, that are the most characteristic, significant, distinct and/or reproducible, have been tabulated, but additional peaks can be extracted, using conventional methods, from the diffractogram. The presence of these main peaks, reproducible and within the error limit, is for most circumstances sufficient to establish the presence of said crystal modification.

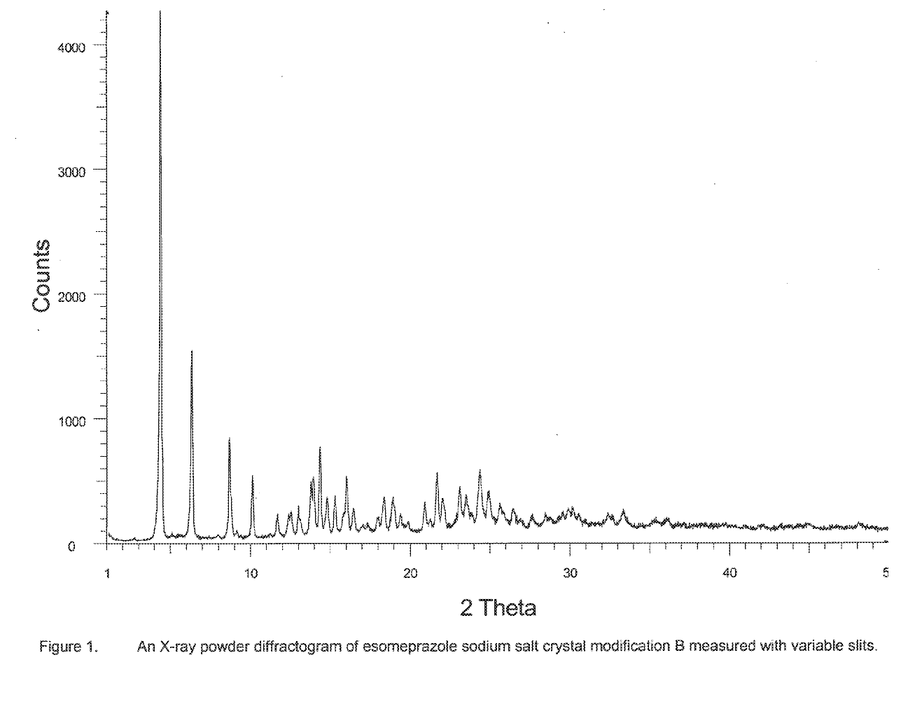

Esomeprazole sodium salt modification B is further characterized in providing an X-ray powder diffraction pattern as essentially shown in FIG. 1.

Esomeprazole sodium salt modification B is a crystalline form exhibiting advantageous properties, such as convenient handling as well as chemical and solid-state stability.

It is possible to crystallize esomeprazole sodium salt modification B, i.e. the compounds of the present invention in one single solvent or in a mixture of solvents. However, it is preferred that the crystallization is from water.

Crystallization may also be initiated and/or effected with or without seeding with crystals of the appropriate crystalline compound of the invention.

Crystallization of compounds of the present invention can be achieved starting from pure esomeprazole sodium salt of any form, or mixtures of any forms.

One object of the present the invention is to provide processes for the preparation of Esomeprazole sodium salt modification B.

Esomeprazole sodium salt modification B is obtained upon crystallization from water.

In order to ensure that a particular crystalline form is prepared in the substantial absence of other crystalline forms, crystallization is preferably carried out by seeding with seed crystals of the desired crystalline form.

Esomeprazole sodium salt modification B obtained according to the present invention is substantially free from other crystal and non-crystal forms of esomeprazole sodium salt. The term “substantially free from other crystal and non-crystal forms esomeprazole sodium to salt” shall be understood to mean that the desired crystal form of esomeprazole sodium salt contains less than 15%, preferably less than 10%, more preferably less than 5% of any other forms of esomeprazole sodium salt.

The crystal modifications of the present invention are effective as a gastric acid secretion inhibitor, and are thus useful as antiulcer agents. In a more general sense, they can be used for prevention and treatment of gastric-acid related conditions in mammals and especially in man, including e.g. reflux esophagitis, gastritis, duodenitis, gastric ulcer and duodenal ulcer. Furthermore, they may be used for treatment of other gastrointestinal disorders where gastric acid inhibitory effect is desirable e.g. in patients on NSAID therapy, in patients with Non Ulcer Dyspepsia, in patients with symptomatic gastro-esophageal reflux disease, and in patients with gastrinomas. They may also be used in patients in intensive care situations, in patients with acute upper gastrointestinal bleeding, pre- and postoperatively to prevent aspiration of gastric acid, to prevent and treat stress ulceration and asthma, and for improvement of sleep. Further, the crystal modifications of the invention may be useful in the treatment of psoriasis as well as in the treatment of Helicobacter infections and related diseases. The crystal modifications of the invention may also be used for treatment of inflammatory conditions in mammals, including man.

Any suitable route of administration may be employed for providing the patient with an effective dosage of the crystal modifications. For example, peroral or parenteral formulations, including i.v., and the like may be employed. Dosage forms include capsules, tablets, dispersions, suspensions, solutions and the like.

It is further provided a pharmaceutical composition comprising the crystal modifications of the present invention, as active ingredient, in association with a pharmaceutically acceptable carrier, diluent or excipient and optionally other active pharmaceutical ingredients. Compositions comprising other therapeutic ingredients are of interest in the treatment of the conditions listed above. The invention also provides the use of the crystal modifications in the manufacture of a medicament for use in said conditions as well as a method of treating a gastric-acid related condition which method comprises administering to a subject suffering from said condition a pharmaceutically effective amount of the crystal modifications.

The compositions of the invention includes compositions suitable for peroral, i.v. or other parenteral administration modes. The most preferred route is the i.v. route. The compositions may be conveniently presented in unit dosage forms, and prepared by any methods known in the art of galenic pharmacy.

In the practice of the invention, the most suitable route of administration as well as the magnitude of the therapeutic dose will depend on the nature and severity of the disease to be treated. The dose, and dose frequency, may also vary according to the age, body weight and response of the individual patient. Special requirements may be needed for patients having Zollinger-Ellison syndrome, such as a need for higher doses than the average patient. Children and patients with liver diseases generally will benefit from doses that are somewhat lower than average. Thus, in some conditions it may be necessary to use doses outside the ranges stated below, for example long-term treatments may request lower dosage. Such higher and lower doses are within the scope of the present invention. Such daily doses may vary between 5 mg to 300 mg.

In general, a suitable oral dosage form of the compound of the invention may cover a dose range from 5 mg to 300 mg total daily dose, administered in one single dose or equally divided doses. A preferred dosage range is from 10 mg to 80 mg.

The compound of the invention may be combined as the active component in intimate admixture with a pharmaceutical carrier according to conventional techniques, such as the oral formulations described in WO 96/01623 and EP 0 247 983, the disclosures of which are hereby as a whole included by reference.

Combination preparations comprising the compounds of the invention and other active ingredients may also be used. Examples of such active ingredients include, but are not limited to anti-bacterial compounds, non-steroidal anti-inflammatory agents, antacid agents, alginates and prokinetic agents.

The compounds of the invention may be further processed before formulation into a suitable pharmaceutical formulation. For example, the crystal modification may be milled or ground into smaller particles.

For the avoidance of doubt, “treatment” includes the therapeutic treatment, as well as the prophylaxis, of a condition.

The presence of additional substances in a sample, like pharmaceutical excipients, to be characterised by X-ray powder diffraction can of course mask some of the small peaks in any of the above characterized crystal modifications. This fact alone can of course not demonstrate that the crystal modification is not present in the sample. Under such circumstances due care must be used and the presence of substantially all main peaks in the X-ray powder diffraction pattern might suffice to characterize the crystal modification. It is thus preferred to analyse the crystal modifications of the present invention without the presence of additional substances. Alternatively, other well-known analytical methods may be used to characterize the crystal modifications, e.g. Raman spectroscopy.

According to a further aspect of the invention there is provided a method of treatment of a condition where esomeprazole sodium salt is required or desired, which method includes administering a therapeutically effective amount of a compound of the invention to a patient in need of such treatment.

The compounds of the invention have the advantage that they are in a form that provides for improved ease of handling. Further, the compounds of the invention have the advantage that they may be produced in forms that have improved chemical and solid state stability as well as lower hygroscopicity. Thus, the compounds may be stable when stored over to prolonged periods.

The invention is illustrated, but in no way limited, by the following examples.

### EXAMPLES

General

X-ray powder diffraction analysis (XRPD) was performed on samples prepared according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray analyses were performed using a Siemens D5000 diffractometer and/or a Phillips X'Pert MPD using Cu Ka radiation.

XRPD distance values may vary in the range ±2 on the last decimal place.

### Example 1

### Preparation of Esomeprazole Sodium Salt Modification B

50 mg esomeprazole sodium salt was put in a vial and 10 mg of water was added. The vial was closed and the mixture was stirred and left overnight whereafter the title compound was isolated.

The crystals were analyzed by XRPD and the results are tabulated below (Table 1) and are shown in FIG. 1.

Modification B

| d-value | Relative |
|---|---|
| (Å) | intensity |
| 20.4 | vs |
| 14.0 | vs |
| 10.2 | s |
| 10.1 | m |
| 9.7 | w |
| 8.8 | s |
| 7.6 | m |
| 7.2 | m |
| 7.1 | m |
| 6.8 | m |
| 6.8 | m |
| 6.4 | m |
| 6.4 | s |
| 6.2 | s |
| 6.0 | m |
| 5.8 | m |
| 5.6 | m |
| 5.5 | m |
| 5.4 | m |
| 5.2 | w |
| 5.1 | w |
| 4.94 | w |
| 4.830 | m |
| 4.69 | m |
| 4.58 | m |
| 4.47 | w |
| 4.24 | m |
| 4.18 | w |
| 4.10 | s |
| 4.03 | m |
| 3.84 | m |
| 3.78 | m |
| 3.13 | W |
| 3.65 | m |
| 3.57 | m |
| 3.47 | m |
| 3.36 | w |
| 3.31 | w |
| 3.23 | w |
| 3.14 | w |
| 3.02 | w |
| 2.99 | w |
| 2.96 | m |
| 2.93 | w |
| 2.76 | w |
| 2.68 | w |
| 2.48 | w |

The peaks, identified with d-values calculated from the Bragg formula and intensities, have been extracted from the diffractogram of esomeprazole sodium salt form A, shown in FIG. 1. The relative intensities are less reliable and instead of numerical values the following definitions are used;

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with variable slits.

### Reference Example Prepared According to Example of WO 96/02535

### Asymmetric Synthesis of (−)-5-methoxy-2-(((4-methoxy-3,5-dimethyl-2-pyridinyl)-methyllsulphinyl]-1H-benzimidazole sodium salt, (−)-Na salt

59 g (180 mmol) of 5-methoxy-2-[[(4-methoxy-3,5-dimethyl-2-pyridinyl)-methyl]thio]-1H-benzimidazole was dissolved in 200 ml ethyl acetate. To the solution was added 0.3 ml (17 mmol) water. To the mixture was added 37 g (180 mmol) (+)-diethyl L-tartrate, 25 g (90 mmol) titanium(W) isopropoxide and 16 ml (90 mmol) diisopropylethylamine at room temperature. The addition of 30 ml (160 mmol) cumene hydroperoxide (80%) was then performed over a period of 90 minutes at 34° C. After cooling to room temperature for 120 minutes a small sample of the mixture was taken for chiral and achiral chromatographic analyses.

The mixture consisted of 82% sulphoxide with an enantiomeric excess (e.e.) of 87%. The mixture was diluted with 60 ml isooctane and 40 ml ethyl acetate whereupon the product was extracted three times with an aqueous ammonia (12%) solution with a total volume of 480 ml. The combined aqueous phases were neutralised by addition of 50 ml concentrated acetic acid. Thereafter, the workup procedure employed extraction, evaporation, sodium hydroxide addition and crystallisation procedures yielding 32.7 g of the title compound with a purity of 95.2% (achiral analysis) and with an enantiomeric excess (e.e.) of 99.8% (chiral analysis). The overall yield was 47.2%.

1. Esomeprazole sodium salt crystal modification B, characterized in providing an X-ray powder diffraction pattern exhibiting substantially the following main peaks with d-values; 20.4, 14.0, 10.2, 8.8, 6.2, and 4.10 Å.

2. Esomeprazole sodium salt modification B according to claim 1, characterized in providing an X-ray powder diffraction pattern as essentially shown in FIG. 1.

3. A pharmaceutical formulation comprising esomeprazole sodium salt modification B as defined in claim 1 in admixture with at least one pharmaceutically acceptable excipient.

4. The use of esomeprazole sodium salt modification B as defined in claim 1 in therapy.

5. The use of esomeprazole sodium salt modification B as defined in claim 1 as active ingredient in the manufacture of a medicament for use in treatment of gastrointestinal inflammatory diseases.

6. A method of treatment of gastrointestinal inflammatory diseases which comprises administration of a therapeutically effective amount of esomeprazole sodium salt modification B as defined in claim 1, to a patient suffering therefrom.
